(19) United States
(12) Patent Application Publication
(10) Pub. No.: US 2012/0053423 A1
(43) Pub. Date: Mar. 1, 2012

(54) SMART MATTRESS

(76) Inventors: Christopher Kenalty, Toronto (CA); Miriam Gordon, Toronto (CA)

(21) Appl. No.: 12/862,253

(22) Filed: Aug. 24, 2010

(51) Int. Cl. A61B 5/00

(52) U.S. Cl. 600/300

(57) ABSTRACT

Systems and methods are disclosed for a self-contained Smart Mattress which is capable of monitoring the vital statistics of a patient in real time, analyzing the data using an embedded processor, storing patient identification and information, producing an electronic medical report and communicating any data to the caregiver or computer server using wireless technology, such as Bluetooth® Technology. Due to the self-contained nature of the mattress, it is also ideal for use as an emergency evacuation mattress for bed-ridden patients.

### TECHNICAL FIELD

The present invention relates to a Smart Mattress for both hospital and bed-ridden patients. More particularly, the invention relates to such a mattress which is capable of measuring and reporting a patient's vital statistics to a server or caregiver during emergency and non-emergency situations.

### BACKGROUND INFORMATION

There are a number of existing methods and apparatus which are capable of providing for real-time monitoring of a patient's vital statistics. These apparatus include electrocardiogram recorders, heart rate monitors, blood pressure monitors, electroencephalograph apparatus, pulse oximetry meters, carbon dioxide meters, thermostats, scales, maternal uterine activity monitors, and various other noninvasive medical instrumentation.

A major concern with current noninvasive medical instruments is that they are often bulky and require an excessive number of cables in order to report the measurements to a computer or physician. Similarly, in emergencies, non-ambulatory or bed-ridden patients must be quickly and safely evacuated from hospitals, a situation in which patients wait (often for hours) for transportation to another hospital. In such emergencies, because of the issues mentioned above, most current medical instrumentation cannot be quickly and effectively packed up to preserve substantially uninterrupted patient monitoring. Similarly, in the case of home health care, it would be highly desirable to simplify and/or create more user-friendly monitoring methods by reducing the amount of equipment and creating a self-contained, maintenance-free monitoring device.

Numerous improvements in the medical field have been made to reduce the number of monitors physically attached to a patient and the size and number of devices in a hospital room by integrating certain sensor devices into the existing bedding of a patient. The following patents and patent publications, which are hereby incorporated by reference in their entirety herein, disclose a number of contactless, non-invasive patient monitoring methods.

A mattress pad disclosed in U.S. Patent Publication No. 20070149883 to Yesha has at least two pressure-sensitive piezoelectric sensors positioned in a rigid pad beneath the patient's mattress. The mattress pad includes a processor to receive all sensor measurements and calculate heart and respiration rates, which are determined by subtracting the pressure signals corresponding to the upper body and the lower body of a patient and mathematically determining the maximum difference of signal between each group of sensors. The heart and respiration rates are then transmitted by a cable to existing patient monitoring equipment. This system, however, requires that a rigid pad be properly disposed and positioned beneath the mattress while physically connected to an auxiliary device, restricting both the mobility and versatility of the monitoring system.

A mattress with integrated piezoelectric sensors disclosed in U.S. Pat. No. 7,652,581 to Gentry has a passive sensor, or sensor array, in the mattress pad that supports continuous monitoring of a patient's physiological condition in a hospital setting. The external processor receives sensor data, either by wired or wireless communication, from mattress pad sensors and processes sensed data into a form that is usable by a physician, nurse or other user. As in the Yesha reference, this is not a self-contained monitoring mattress and still requires auxiliary equipment to receive and process sensor data.

Yet another monitoring mattress pad apparatus is disclosed in U.S. Pat. No. 7,164,941 to Misczynski. In this reference, a contactless electromagnetic inductance device collects cardiac activity signals to evaluate patient sleep. As in the prior references, the system is not self-contained and also does not permit storage of patient data.

There are numerous other patents and patent applications which employ a pad or embedded mattress sensor coupled to an auxiliary device. One apparent disadvantage is lack of a self-contained system and the reliance on or requirement of an auxiliary device. Another disadvantage is the non-obvious embedded storage for patient identification and other data, such as a patient's chart information.

Thus, what is needed is a self-contained Smart Mattress which is capable of monitoring the vital statistics of a patient in real time, analyzing data using an embedded processor, storing patient identification and information, producing an electronic medical report and communicating any data to a caregiver or computer server using wireless technology, such as Bluetooth® Technology. Due to the self-contained, portable nature of the Smart Mattress and Smart Mattress technology, it is also ideal for use with an emergency evacuation mattress system for hospital patients and other bed-ridden patients.

### SUMMARY

Health care providers are very mobile, and the adoption of enhanced wireless technology by health care organizations, especially when harnessed properly, can help to improve, even automate, patient care and monitoring. It is indisputable that computer networks are commonplace in health care organizations and in some places are indispensable. However, most of the computer devices and monitoring devices are connected through the use of wires; this usually means that their use is limited to a fixed place. A Smart Mattress eliminates many of the wires associated with current methods and allows for greater flexibility in patient monitoring.

For this application the following terms and definitions shall apply:

The terms “communicate” and “communicating” as used herein include both conveying data from a source to a destination and delivering data to a communications medium, system, channel, network, device, wire, cable, fiber, circuit and/or link to be conveyed to a destination, and the term “communication” as used herein means data so conveyed or delivered. The term “communications” as used herein includes one or more of a communications medium, system, channel, network, device, wire, cable, fiber, circuit and link.

The term “processor” as used herein means processing devices, apparatus, programs, circuits, components, systems and subsystems, whether implemented in hardware, tangibly embodied software or both, and whether or not programmable. The term “processor” as used herein includes, but is not limited to, one or more computers, hardwired circuits, signal modifying devices and systems, devices and machines for controlling systems, central processing units, programmable devices and systems, field-programmable gate arrays, application-specific integrated circuits, systems on a chip, systems comprising discrete elements and/or circuits, state machines, virtual machines, data processors, processing facilities and combinations of any of the foregoing.

The present disclosure endeavors to provide a Smart Mattress and/or Smart Mattress system capable of monitoring the vital statistics of a patient in real time, analyzing the data using an embedded processor, storing patient identification and information, producing an electronic medical report and communicating any data to a care provider, computer server or portable device using wireless technology. A portable device may be a cell phone, smart phone, Personal Digital Assistant (“PDA”), media player/reader, computer laptop, tablet PC, or any other processor-based device that is known in the art, including a desktop PC and computer workstation. Using a portable device allows for more flexibility when monitoring a patient or during emergency evacuations by allowing the health care provider to monitor from a distance or to receive automatic alerts when a patient's condition changes.

Integrating a Smart Mattress with a hand-held device or an emergency evacuation system has not been done. Use of a Smart Mattress alone or in combination with a portable device will enhance data collection during both day-to-day monitoring and in emergency evacuations. Use of a Smart Mattress improves data accuracy, reduces paperwork, supports collection of more complete information, eliminates redundant data entry, allows faster adaptation to changing conditions and provides access to previously unavailable information. Additionally, when a patient is transferred to an alternate facility, patient records, which may be stored in a Smart Mattress DSU, travel with the patient.

### BRIEF DESCRIPTION OF THE DRAWINGS

FIG. 1 is a high-level diagram illustrating a first embodiment for using Smart Mattress;

FIG. 2 is a diagram illustrating a first embodiment for using Smart Mattress;

FIG. 3 is a diagram illustrating a second embodiment for using Smart Mattress;

FIG. 4 is an exemplary screen shot of the portable device;

FIG. 5 is a diagram illustrating a portable device; and

FIG. 6 is a diagram illustrating an exterior embodiment of a Smart Mattress.

### DETAILED DESCRIPTION

A preferred embodiment of the present invention will be described herein below with reference to the accompanying drawings. In the following description, well-known functions or constructions are not described in detail since they would obscure the invention in unnecessary detail.

A Smart Mattress as described herein would include a data storage unit (“DSU”) for storing patient information (e.g., demographics such as name, height, weight, gender, date of birth, race, religion, blood type and other patient particulars), physiological sensors for automatically updating the patient's vital statistics (e.g., body temperature, systolic and diastolic blood pressures, pulse, respiratory rate, pulse oximetry and electrocardiogram), and a processor for handling, processing and/or time-stamping any updates to a DSU. The sensors may be embedded in the mattress or directly coupled to the patient. When the sensor is coupled to a patient, the Smart Mattress may wirelessly communicate information with the sensor using short-range wireless communication, e.g., Bluetooth technology. Alternatively, the Smart Mattress may include ports where additional wired sensors may be connected by the user.

In a preferred embodiment, a Smart Mattress includes an on-board power source, such as a rechargeable battery, to increase the portability of a Smart Mattress. The rechargeable battery may be recharged using the standard AC wall current or the battery may be removed and charge using a dock or charging station. Alternatively, a Smart Mattress may be capable of receiving power directly from an auxiliary power source (e.g., wall socket, solar panel or other generator).

A DSU is capable of collecting and disseminating patient information from initial patient assessment through disposition (to provide an electronic medical record), including triage, treatment and transport of patients for daily and mass casualty operations. To better prepare for disasters, major hurricanes, earthquakes, terrorist acts, mass casualty events and disease epidemics, a DSU may link health providers with on-scene responders and track patients from first assessment through triage, treatment and transport. A DSU may also store information regarding any medications and dosages (including intravenous fluids, morphine and other medications), treatment status, disposition of treatment and physician and include a time stamp of the last update.

Sensors or other monitoring devices should be in constant proximate contact with a patient, allowing for continuous updates to the data in a mattress's DSU. A Smart Mattress may also be capable of simultaneously updating a medical facility's information system and a provider's or first responder's portable device with a patient's vital sign information. Simultaneously updating a medical facility's information provides a backup in the event of data loss in a Smart Mattress.

Updates regarding a patient's information may be communicated using wireless technology between a medical facility, health care provider or portable device and a DSU in a Smart Mattress. Similarly, if patient information (e.g., vitals or health condition) deviates from an established range or value, that information may also be wirelessly communicated to a health care provider either directly from a Smart Mattress or via a computer network.

Two more popular types of wireless technology standards available are Bluetooth® and the Institute of Electrical and Electronic Engineering's (IEEE) 802.11 standards (“Wi-Fi”). Bluetooth® is an open specification delivering short-range radio communication between electrical devices that are equipped with Bluetooth® chips. When two Bluetooth®-enabled devices are within communication range (presently about 10 meters), they send each other a unique ID to identify one another. This ID is used to determine the type of information to be shared and the level of functionality that could occur between the two devices. However, Bluetooth® is not designed for long-distance communication but rather as a means for providing connections between mobile computing devices or between a mobile computer device and a hub. To increase operating range, an potential solution would be to couple Bluetooth® technology with Wi-Fi, which has a larger operating range of up to 300 meters. Wi-Fi is an extension of the wired Ethernet and uses the same principles as its wired counterpart, thus providing its users with high-speed, reliable connections to a network. Alternatively, a Bluetooth® range extender may be integrated into the system to enhance the communication range and to eliminate the need for Wi-Fi.

A medical provider may utilize a portable device to access and update patient information stored in a Smart Mattress. Using a portable device allows for more flexibility when monitoring a patient or during emergency evacuations by allowing the health care provider to monitor from a distance or to receive automatic alerts when a patient's condition changes. Since patient vitals and demographic information are stored in a Smart Mattress's DSU, there is no need to input this information when updating a patient's status and disposition during relocation or evacuation. A Smart Mattress DSU also eliminates the need for a paper-based system both during emergency evacuations and in general patient monitoring. Medical staff or health care providers are able to conduct a reassessment of a patient's condition and upload those comments to the DSU in a mattress using a portable device. The time of a reassessment may also be automatically time-stamped in order to track a patient's condition. A provider will additionally be able to note what time a patient arrived at a certain location.

A portable device allows a care provider to review patient information from a distant location, make a diagnosis and upload that information to a DSU in a Smart Mattress via wireless or wired communication. Additionally, if a health care provider feels a patient needs immediate medical care, the provider may order certain actions that would be sent to on-site personnel, a medical unit or an evacuation site. Provider-directed actions could also be sent directly to a staff member's portable device. Ideally, the hospital or evacuation site would have a monitoring station that is capable of communicating information with a Smart Mattress, similar to an intensive care unit's nursing station which monitors patient information, enabling quick medical decisions.

A Smart Mattress is ideal for use as an emergency evacuation mattress for bed-ridden patients. This is due in part to a Smart Mattress being self-contained, capable of monitoring vital statistics of a patient in real time, analyzing data using an embedded processor, storing patient identification and information, producing an electronic medical report and communicating any data to the caregiver or computer server using wireless technology, such as Bluetooth® Technology. Integrating Smart Mattress functionality (e.g., the features of a Smart Mattress) with an evacuation mattress, such as the Evacusled, is a life-saving novel combination.

An Evacusled, or evacuation mattress, is disclosed in U.S. Patent Publication No. 2008/0301876 to Christopher Kenalty, which is assigned to the assignee of the present application, and which is hereby incorporated by reference in its entirety herein. Evacusled teaches an emergency evacuation mattress for bed-ridden patients that is capable of operation by a single caregiver, provides a warm and secure cocoon for a patient, allows easy transport over any type of surface, provides proper support for all of a patient's body and bedding, and allows a patient to feel a high degree of comfort in what is otherwise a very stressful situation. The Evacusled would be an ideal candidate for integration with the Smart Mattress and/or Smart Mattress functionality.

Several other advantageous evacuation devices and techniques are also disclosed in U.S. Pat. No. 5,249,321 to Jorg Graf and U.S. patent Ser. No. 12/700,027, filed Feb. 4, 2010, to Christopher Kenalty, which are both assigned to the assignee of the present application, and which are incorporated by reference herein.

Another area of concern during patient evacuation is accurate tracking of a patient and mattress. This may be solved by utilizing Radio-Frequency Identification (or “RFID”) tags embedded inside a Smart Mattress, enabling real-time location and movement information which can be sent to remote monitoring equipment or medical staff during emergency evacuation—ensuring a facility has not left anyone behind.

A Smart Mattress, either alone or in combination with the Evacusled, should be capable of providing all of these novel features while still providing the comfort and safety features usually associated with traditional and hospital mattresses. For example, a Smart Mattress should be designed to reduce the potential for pressure ulcers. This can be accomplished using embedded sensors in the mattress that would map pressure points and alert medical staff when an area exceeds the established threshold. All pressure point information may also be stored in an embedded DSU.

A Smart Mattress should include a cover (e.g., nylon, vinyl, plastic) to help reduce the likelihood of fluid penetration and prevent damage to electronic devices stored in the mattress. Having a firmer perimeter foam built into a Smart Mattress could help keep a patient centered on the mattress at all times and therefore reduce the risk of rolling out of bed. Additionally, the underside of a mattress may have fasteners, such as magnets or hooks and loops (e.g., VELCRO), to help keep the bed linens in place and keep the mattress from sliding or shifting. The Smart Mattress and Evacusled components could be radiolucent to allow x-rays to pass through them. The existing Evacusled uses metal axles for the underside wheels which may prevent the use of a bedside fluoroscopy. A critical care bed should have a radiolucent platform attached to the bed deck and a radiolucent mattress, or Smart Mattress, to allow the use of fluoroscopy at the bedside. To allow for bedside x-ray use, the Smart Mattress and Evaculsed combo should both be radiolucent, a feature that, due to its versatility and ease of use, will be well-received in the critical care bed market.

A potential alternative to creating a 100% radiolucent Smart Mattress and Evacusled combo is to create an x-ray cassette sleeve in the side of the mattress. The side opening allows for easy insertion of an x-ray cassette. The sleeve could be located under the top of the mattress (e.g., approximately 1 inch deep) so as not to be in direct contact with the patient. This method would increase patient and caregiver safety and reduce the chances of injury associated with portable x-ray procedures.

During daily use, the external skin of the Smart Mattress may become dirty, damaged or torn, especially when the Smart Mattress is used during an evacuation procedure, where the underside (e.g., adjacent to where wheels may be located on a traditional Evacusled) may easily become damaged due to abrasions from transport. Due to the costs of mattresses, including the Smart Mattress, it would be advantageous to provide a replacement skin that a hospital could purchase for the Smart Mattress or Smart Mattress/Evacusled combo rather than having to buy an entirely new system.

The top skin and bottom skin panels may be zipped together and secured with a flap to form a continuous skin. In another embodiment, the skin may also include an intermediate side-wall skin between the top and bottom skin panels. The skin material should meet infection control measures and may also contain microclimate features. The replacement skins would retail for just a fraction of the cost of the Smart Mattress, therefore enhancing the life of the Smart Mattress and/or Evacusled.

The Smart Mattress and/or Evacusled should meet the demanding infection control measures which are essential in medical facilities. A solution to maintain an anti-fugal and anti-bacterial mattress surface would be to coat the mattress system with spray-on liquid glass. Spray-on liquid glass is transparent, non-toxic, and can protect virtually any surface against almost any damage from hazards such as water, UV radiation, dirt, heat, and bacterial infections. Liquid glass coating is also flexible and breathable, which makes it suitable for use on hospital mattresses.

Liquid glass spray (also referred to as “SiO2 ultra-thin layering”) consists of almost pure silicon dioxide (silica, the normal compound in glass) extracted from quartz sand. Water or ethanol is added, depending on the type of surface to be coated. There are no additives, and the nano-scale glass coating bonds to the surface because of the quantum forces involved. Liquid glass has a long-lasting antibacterial effect because microbes landing on the surface cannot divide or replicate easily.

Liquid glass spray produces a water-resistant coating only around 100 nanometers (15-30 molecules) thick. On this nano-scale the glass is highly flexible and breathable. Liquid glass coating is environmentally harmless and non-toxic, and easy to clean using only water or a simple wipe with a damp cloth. It repels bacteria, water and dirt, and resists heat, UV light and even acids. Food processing companies in Germany have already carried out trials of the spray, and found sterile surfaces that usually needed to be cleaned with strong bleach to keep them sterile needed only a hot water rinse if they were coated with liquid glass. The levels of sterility were higher for the glass-coated surfaces, and the surfaces remained sterile for months. A year-long trial of the spray in a Lancashire hospital also produced very promising results for a range of applications including coatings for equipment, medical implants, catheters, sutures and bandages.

Since many patients spend a majority of their hospital stays on mattresses, and, depending on their conditions, may spend their entire lives confined to their beds, the Smart Mattress and Evacusled must combine the best practices of therapeutic mattress design.

Depending on the patient's need, there may be various Smart Mattress models. A standard Smart Mattress model may include a form mattress and Evacusled absent additional therapeutic features. A medical bed with air would integrate an air surface mattress to prevent pressure ulcers and may contain multiple independent zones of continuous low pressure to reduce the peak pressures that cause and aggravate skin ulcers. Yet another example may be a critical care design to be used in intensive care wards. A critical care design would be radiolucent or contain the x-ray cassette sleeve stated above. Additionally, a critical care mattress may have the most advanced therapeutic feather currently available in the marketplace (e.g., pressure redistribution surface, microclimate mattress surface to remove heat and moisture to cool the patient and keep the patient's skin drier, weight-based pressure sensors to distribute patient's weight, and a patient turn-assist feature to make it easy to change linens and conduct skin assessments on bed-ridden, critical care patients). Naturally, various combinations of these models may be made depending on the market's demand.

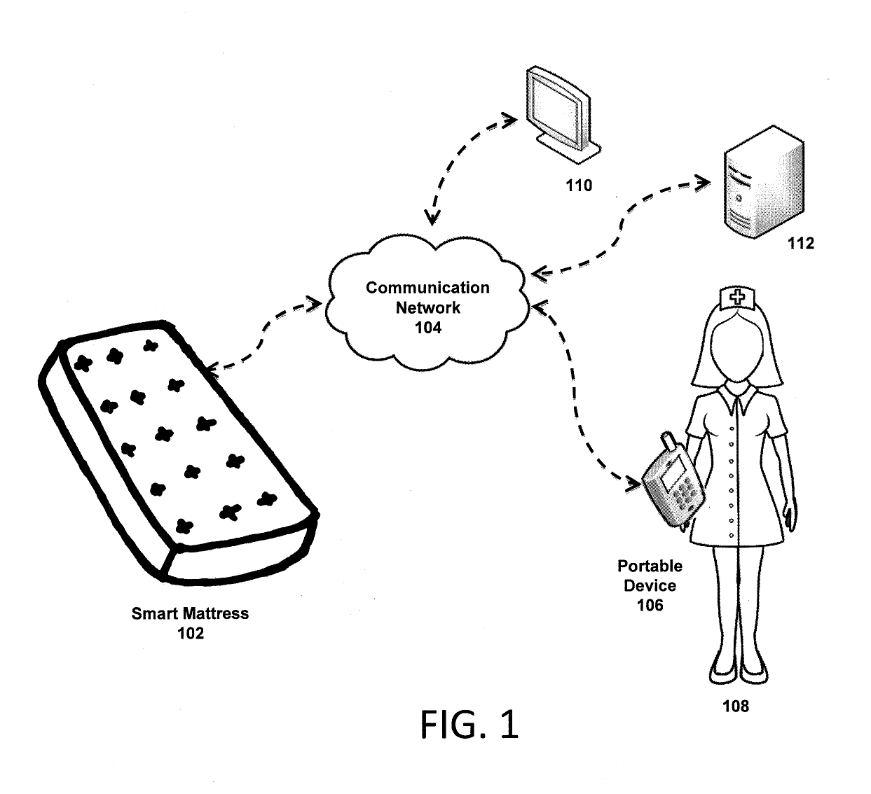

Referring now to FIG. 1, a Smart Mattress 102 system is depicted. The Smart Mattress 102 system of FIG. 1 uses a communication network 104 to wirelessly transmit a patient's physiologic data or dynamic patient data to portable device 106, monitoring station 110 or other computer server 112. The portable device 106 may be carried by a health care provider 108 or other personnel interested in the health of a patient. A monitoring station 110 may be either a local in-hospital nurses' station or a remote monitoring station. Alternatively, patient data may be communicated to a computer server 112 which may be capable of automatically monitoring a patient's health state or forwarding dynamic patient data to one or more health care personnel if a patient's health condition deviates from a predetermined value. The Smart Mattress 102 would include a data storage unit (“DSU”) for storing patient information (e.g., demographics such as name, height, weight, gender, date of birth, race, religion and blood type), physiological sensors for automatically updating a patient's physiological data (e.g., body temperature, systolic and diastolic blood pressures, pulse, respiratory, pulse oximetry and electrocardiogram), and a processor for handling, processing and/or time-stamping any updates to the DSU. In a preferred embodiment, the Smart Mattress 102 includes an on-board power source, such as a rechargeable battery, to increase the portability of a Smart Mattress. Alternatively, a Smart Mattress may be capable of receiving power directly from an auxiliary power source (e.g., wall socket, solar panel or other generator). The Smart Mattress 102 may also include a call button which would signal to the caregiver 108 that assistance is needed (e.g. via the portable device 106 or monitoring station 110).

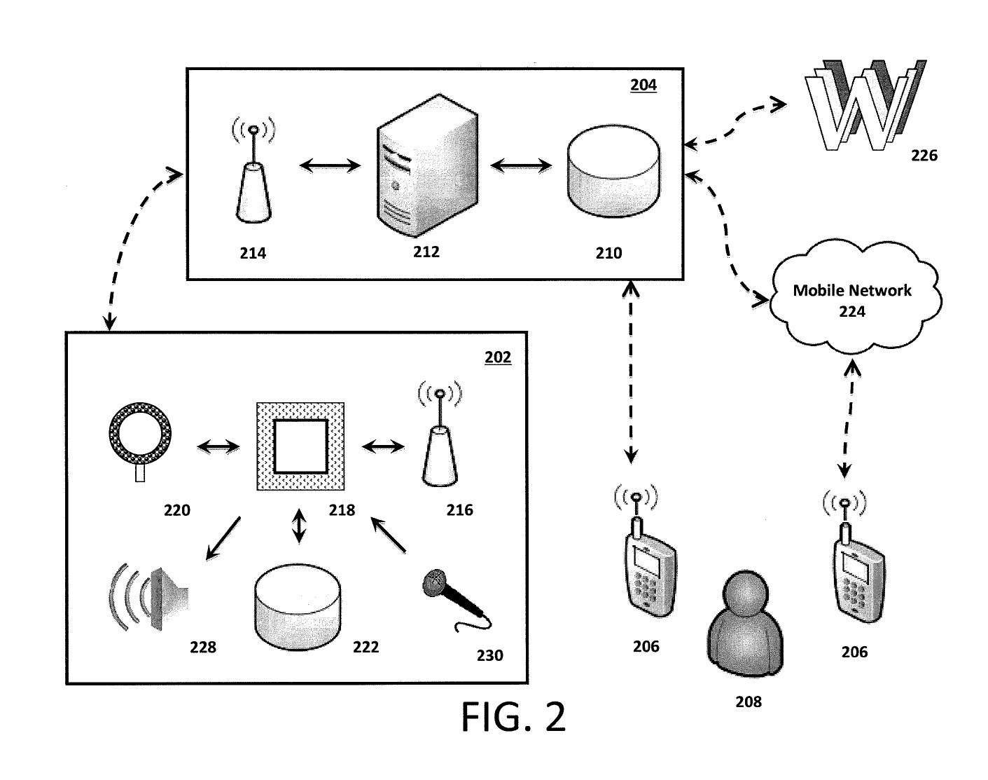

Referring now to FIG. 2, the Smart Mattress system of FIG. 1 is shown in greater detail. The Smart Mattress 202 includes one or more sensors 220, which produce measurement data used in determining a patient's physiological state. The one or more sensors 220 can include pressure sensors, piezoelectric elements, thermometers, insulin detectors, blood-oxygen sensor, weight scales, sensor pad, or a number of other sensors useful in patient monitoring. The processor 218 uses measurement data received from the one or more physiological sensors 220 and/or information stored to the internal memory 222 to produce dynamic patient data. Dynamic patient data may include information such as a patient's physiological data, a patient's identification, doctors' comments or information useful to treatment of a patient. Dynamic patient data may be further time-stamped and stored to the Smart Mattress's 202 DSU 222 or wirelessly transmitted using transmitter 216. The Smart Mattress 202 may also include a microphone 230 and speaker 228 allowing for verbal communication between the Smart Mattress 202 user and portable device 206, monitoring station 110 and/or computer server 112.

The DSU 222 is capable of collecting and disseminating patient information from initial patient assessment through disposition (to provide an electronic medical record), including triage, treatment and transport of patients for daily and mass casualty operations. To better prepare for disasters, major hurricanes, earthquakes, terrorist acts, mass casualty events and disease epidemics, the DSU 222 may link health care providers with on-scene responders and track patients from first assessment through triage, treatment and transport. The DSU 222 may also store information regarding any medications and dosages (including intravenous fluids, morphine and other medications), treatment status, disposition of treatment and physician, and include a time stamp of the last update.

In a preferred embodiment, the Smart Mattress 202 uses Bluetooth® Technology to communicate dynamic patient data to a communication network 204 or a monitoring station (e.g., a nurses' station). The communication network 204 includes a server 212, a wireless transmitter 214 and storage memory 210. The communication network 204 is capable of receiving dynamic patient data from the Smart Mattress 202 via Bluetooth® technology or other wireless means. The communication network 204 is further capable of storing dynamic patient data to the storage memory 210 or wirelessly communicating it to a portable device 206. The communication network 204 may use Bluetooth® technology to communicate with the portable device 206; however, depending on the required range, it may also use another method of wireless communication such as radio frequency communication, microwave communication or infrared (“IR”) short-range communication. The portable device 206 may be carried by a caretaker 208 or other personnel interested in the health of a patient.

In another embodiment, the communication network 204 is further capable of using a mobile network 224 (e.g. a cellular network, GSM, or other public communication channel) to communicate dynamic patient data to a portable devices 206 capable of connecting to mobile network 224 including, but not limited to, a cell phone, smart phone or other hand held portable device. In certain embodiments, various existing portable device's 206 are modified merely by software and/or minor hardware changes to carry out a patient monitoring. In certain other embodiments, portable device's 206 may be redesigned and substantially reconstructed for this purpose. This capability is particularly useful in circumstances, for example, where the primary physician, or care giver, is on holiday or when the monitored patient is at home on bed rest. For example, a doctor attending a conference is Switzerland is able to quickly check the status of his US patients using only his smart phone and without interrupting the patient. Further, if the vitals of a patient deviates from a predetermine range, the doctor, emergency medical personnel and/or any medical staff may be instantly and automatically notified by an alarm, e-mail, call, page, message or the like.

Similarly, dynamic patient data may also be pushed to the Word Wide Web 226 allowing a care giver 208 to access the information from any computer by merely visiting a website and entering their identification information along with any other credentials useful in identifying the user. For security purposes, the website may further require the use of an authentication mechanism such as a piece of hardware (e.g. a token or USB) or software (e.g. a “soft token” for a PDA or cell phone) assigned to a computer user that generates an authentication code or password at fixed intervals. An exemplary authentication mechanism is available from RSA, the security division of EMC, at http://www.rsa.com.

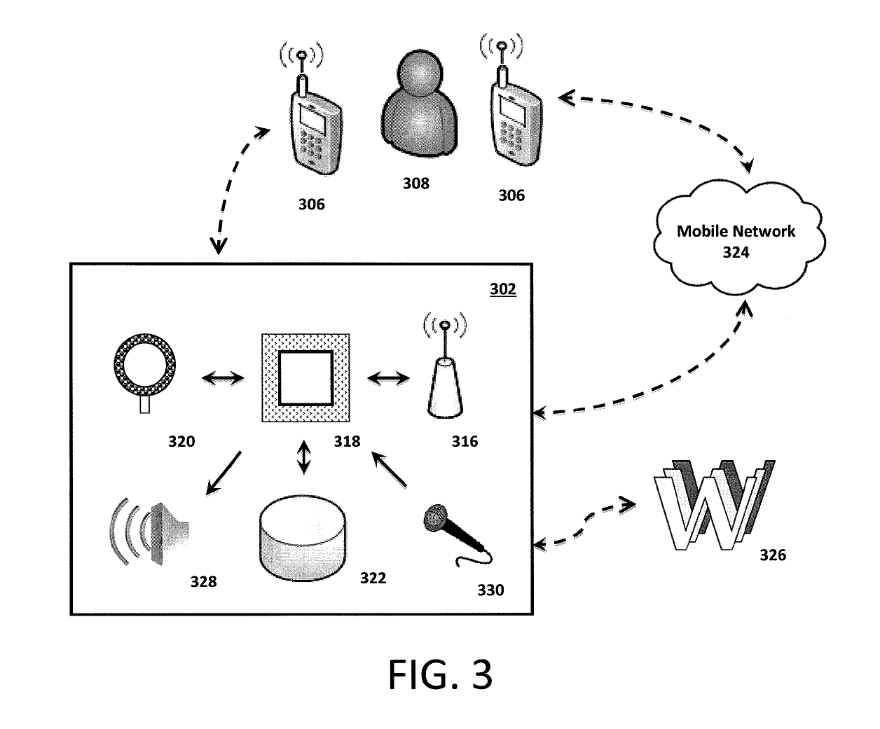

Referring now to FIG. 3, a second embodiment of the present disclosure is shown. As in the first embodiment, the Smart Mattress 302 includes one or more sensors 320, which produce physiological measurement data used in determining a patient's physiological state. The one or more sensors 320 can include pressure sensors, piezoelectric elements, thermometers or a number of other sensors useful in patient monitoring. The processor 318 uses measurement data received from the one or more sensors 320 and information stored to the DSU 322 to produce dynamic patient data. Dynamic patient data may include information such as a patient's physiological data, a patient's identification, doctors' comments or information useful to treatment of a patient. Dynamic patient data may be time-stamped and stored to the Smart Mattress's 302 DSU 322 or wirelessly transmitted using transmitter 316. As distinct from the first embodiment, in the second embodiment the Smart Mattress 302 may use Bluetooth® Technology to communicate dynamic patient data directly to a portable device 306. The portable device 306 may be carried by a caretaker 308 or other personnel interested in the health of a patient. Alternatively, the Smart Mattress 302 may be capable of pushing the dynamic patient data to the World Wide Web 326 or to a mobile network 324 which may transmit the dynamic patient data to one or more portable devices 306.

To eliminate the need for multiple devices, a single portable device 306 may include all of the necessary hardware and/or software necessary to make the portable device capable of communicating with the Smart Mattress 302 in any of the ways describes above.

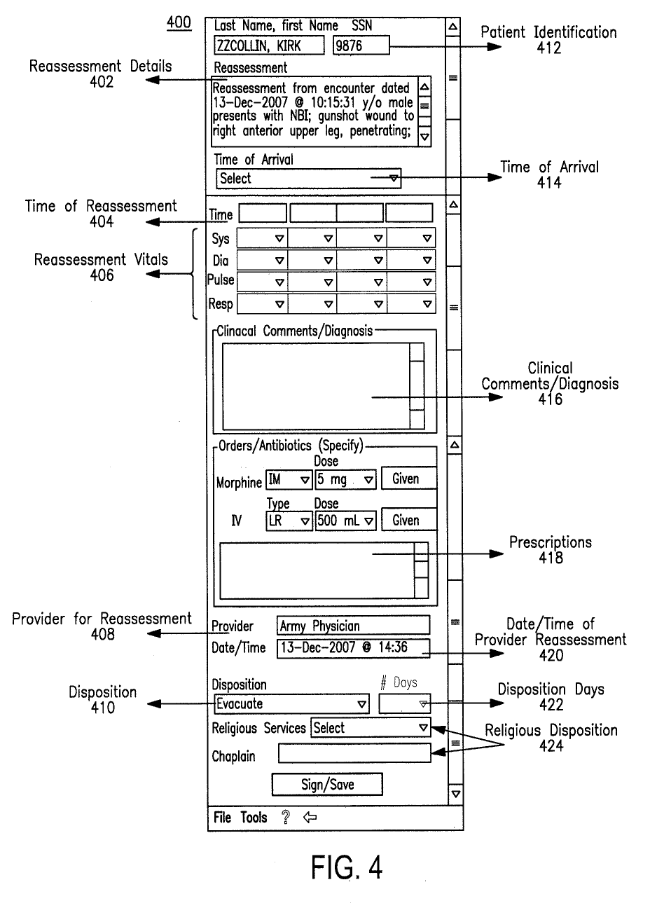

FIG. 4 is an exemplary screen shot of the portable device 400 as disclosed in FIGS. 1, 2 and 3. The portable device 400 is capable of displaying a number of dynamic patient vitals as well as patient data either simultaneously or within a plurality of windows. The portable device 400 contains patient details 412 such as a patient's name, time of arrival 414, social security number, hospital identification number, etc. The portable device is also capable of displaying reassessment vitals 406 (e.g., physiological data) which are measured via a Smart Mattress. In addition to reassessment vitals, the time of reassessment 404 and details of the reassessment 402 may be displayed. These fields would include information regarding any changes to the patient's status and some background on the patient's health, as well as the time of the most recent reassessment and of prior assessments. The portable device 400 also stores information regarding the health care provider or physician responsible or on call for the patient. This provider for reassessment 408 would be displayed along with the time of the most recent or next reassessment 420. Other useful fields are the clinical comments and diagnosis field 416 and prescriptions 418 or current drug/IV rations. The portable device 400 may even provide miscellaneous information such as a patient's religious preference 424 and current disposition 410 and the duration of a patient's disposition 422. Naturally, the portable device would include an interactive means (e.g., a keyboard, touch screen or voice command) to allow a physician or caretaker to input new patient data or comments or to change existing patient data or comments. Any additions or changes to a patient's profile would be communicated to the Smart Mattress, as well as to any intermediate computer servers, so that data stored on the mattress, the portable device and the computer server is constantly synchronized.

In certain embodiments, the portable device 400 may also provide voice communication functionality which allows for real time communication between the patient and the care giver. This may be accomplished using over the air radio, short wave, cellular, GSM, internet or other communication methods known in the art. If the patient or caregiver is unavailable, the portable device 400 and/or Smart Mattress may also be capable of recording voice messages, communicating recorded messages and/or storing/accessing recorded messages from a network (e.g. voicemail).

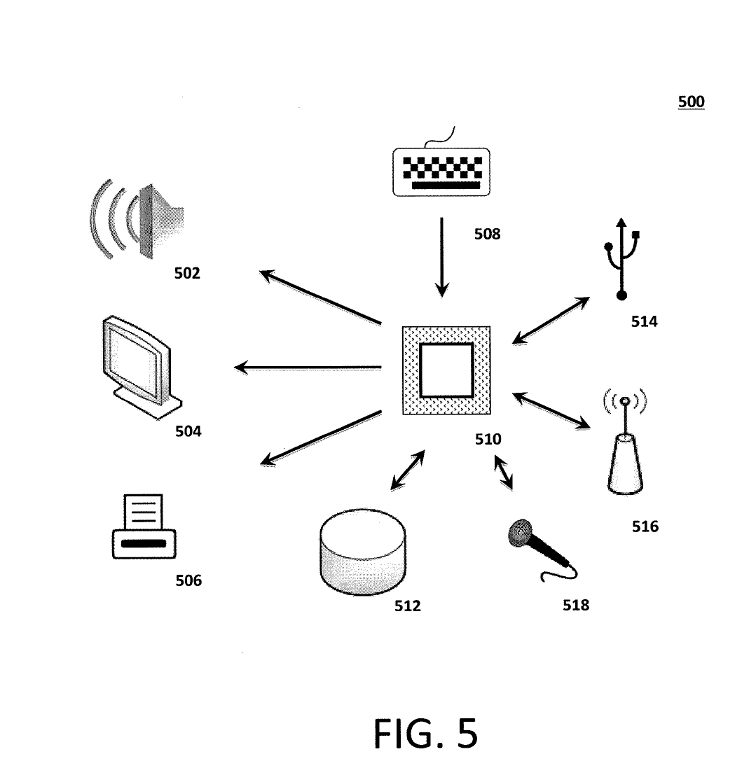

Referring now to FIG. 5, the portable device 500, processor 510, data storage 512 and wireless transmitter 516 are shown. As with the Smart Mattress, the portable device 500 includes a processor 510 and may use Bluetooth® technology or other wireless means to communicate with other devices, including a Smart Mattress, computer network or other devices. Data may be modified or inserted using a manual user interface 508, such as a keyboard, touch screen or voice command. Physiological data, software and any other data or programs may be stored in the data storage memory 512. The data storage memory 512 may be removable or internal. In certain embodiments, the data storage memory 512 may include both an internal memory and removable memory (e.g., flash memory) to allow for easy data transfer and backup. The portable device 500 may also include a wired communication port 514 such as USB or FireWire. The wired communication port 514 allows for connection to and communication with other devices such as a computer. The portable device 500 also includes one or more audio-visual components, such as a screen 504 (e.g., LCD display), one or more speakers 502 and, in certain embodiments, a portable printer 506. The portable printer 506 could be used to print patient medical reports or medical prescriptions on the spot, eliminating the risk of an illegible doctor's note or prescription. The printer may integrated into the portable device or connected by wired or wireless means (e.g. Bluetooth).

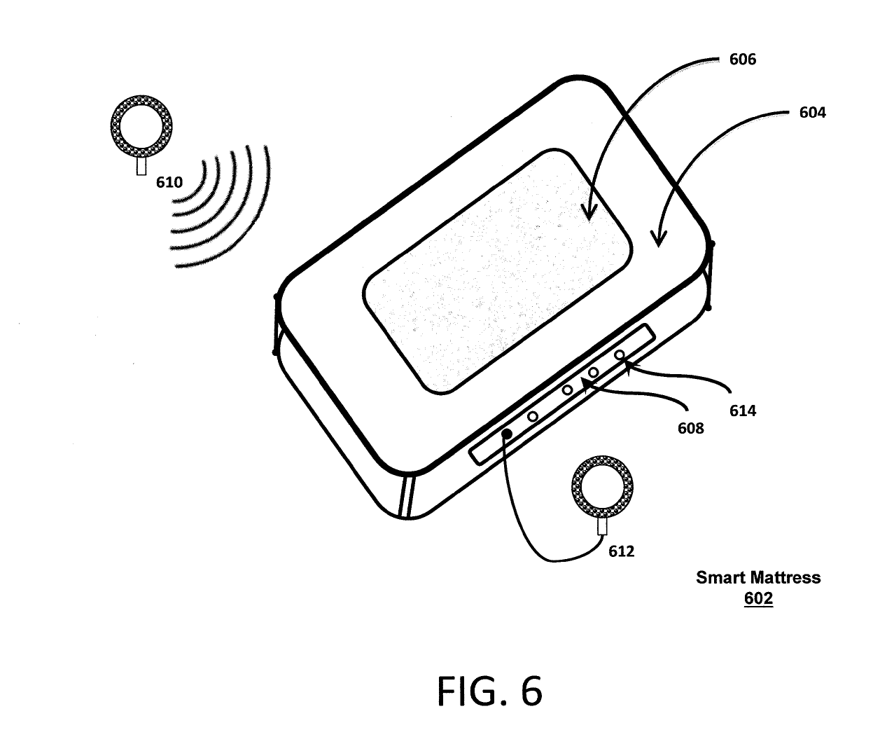

Referring now to FIG. 6., an example diagram of an uncovered Smart Mattress 602 is shown. A Smart Mattress 602 should be capable of providing all of the disclosed monitoring features while still providing the comfort and safety features usually associated with traditional and/or hospital mattresses. The mattress 604 may be constructed from a number of materials (e.g. traditional inner spring, foam, air, gel, water, etc.). The mattress 604 may also have a firmer perimeter to help keep the patient centered on the mattress and in substantially constant contact with one or more sensor pads 606. Additionally, the underside of the mattress 604 may have fasteners, such as magnets or hooks and loops (e.g. VELCO), to help keep bed linens in place and to keep the mattress 604 from sliding or shifting.

The mattress' 604 top surface may include one or more sensor pads 606, or sensor areas, capable of detecting and/or measuring a patient's physiological characteristics from under a sheet and/or blanket and without sacrificing comfort. A single sensor pad 606 is depicted, however a number of sensor pads 606 in various shapes, sizes and sensitivity levels may be used. The one or more sensor pads 606 may be completely integrated with the mattress 604 or detachable to allow easy replacement if damaged. Similarly, the sensor pads 606 may be hard wired to the Smart Mattress 602 or may utilize a detachable plug or clip. In either variation, it would be advantageous to connect each sensor pad 606 such that any sensor cables are not visible. When a patient lies on the Smart Mattress 602, the one or more sensor pads 606 gather physiological characteristic data in real time. The one or more sensor pads 606 may gather data including, but not limited to, temperature, blood pressure, blood-oxygen, weight and heart rate.

An exemplary sensor pad is the Non-Invasive Analysis of Physiological Signals (NAPS) system that was designed and developed at the Medical Automation Research Center at the University of Virginia. The NAPS system pad uses ballistocardiography (BCG) to detect minute forces generated during cardiac contraction and relaxation, and can also detect body movement from respiratory effort and postural changes. The system disclosed in U.S. Patent Publication No. 2005/0124864 to Mack, which is hereby incorporated by reference in its entirety herein, teaches a system for non-invasively detecting, monitoring and analyzing physiological characteristics using a mattress pad. The system relies on a highly sensitive pressure transducer pneumatically connected to a compliant force-coupling pad installed on a mattress.

In certain embodiments, a patient's physiological characteristic may not be easily monitored using a sensor pad 606. In this situation, one or more wireless sensors 610 may be equipped with Bluetooth technology allowing the wireless sensors 610 to communicate directly with the Smart Mattress 602. In the instance where a wired sensor 612 must be used, the Smart Mattress 602 may also include a terminal 608 with a number of wired connectors 614 that allow for the connection of one or more traditional wired sensors 612. Depending on the application, the terminal 608 may contain a single connector type or, to enhance flexibility, a variety of connector types. The terminal 608 may be located on any side of the mattress 604, however, for convenience, it may be advantageous to install the terminal 608 on a mattress 604 side and closer to the head end.

The Smart Mattress may also use a triage tag, which is particularly useful in emergency situations. Triage tags are tools that are often used for first responders and medical personnel use during a mass casualty incident. With the aid of the triage tags, the first-arriving personnel are able to effectively and efficiently distribute the limited resources and provide the necessary immediate care for the victims until more help arrives. Simple Triage and Rapid Treatment (“START”) is a strategy that the first responders and medical personnel employ to evaluate the severity of injury of each victim as quickly as possible and tag a victim in about 30-60 seconds. The triage tags are placed near the head and are used to better separate the victims so that when more help arrives, the patients are easily recognizable for the extra help to ascertain the most dire cases.

For the purpose of the Smart Mattress, Evacusled or combination Smart Mattress Evacusled design, the triage tags will be placed on the foot-end of the mattress, so once the mattress is deployed, the triage tag is clearly visible. The triage tag may have a folding design allowing effective, quick and simple triage, but more importantly the folding tag allows patients to be re-triaged without having to replace the tag. This is in line with States that have standardized triage tags. Presently, the U.S. states and cities that use a standardized or Dynamic Triage Tag include New York, Connecticut, Indiana, Illinois, North Carolina, Nevada, Philadelphia, and Boston. The basic sections of a triage tag include four colors of triage including: Black (Expectant) which entails pain medication only until death; Red (Immediate) which entails life threatening injuries; Yellow (Delayed) which entails non-life threatening injuries; and Green (Minor) which entails minor injuries.

The triage tag may also include a section informing medical personnel of the patient's vital signs along with the treatment administered, a section on the patient's demographics (i.e., gender, residential address, etc. and the patient's medical history), and/or a section with a full pictorial view of the human body where the medical personnel may indicate which parts of the body are injured.

Although various embodiments have been described with reference to a particular arrangement of parts, features and the like, these are not intended to exhaust all possible arrangements or features, and indeed many other embodiments, modifications and variations will be ascertainable to those of skill in the art.

What is claimed is:

1. A Smart Mattress comprising:
one or more physiological sensors;
a data storage unit;
a wireless transmitter to wirelessly communicate patient data to an auxiliary device.

2. The Smart Mattress of claim 1 wherein the auxiliary device is a portable device.

3. The Smart Mattress of claim 1 wherein the auxiliary device is a computer network.

4. The Smart Mattress of claim 1 wherein the auxiliary device is a monitoring station.

5. The Smart Mattress of claim 1 wherein short-range radio communication is used to wirelessly communicate patient data to an auxiliary device.

6. The Smart Mattress of claim 1 wherein the Smart Mattress is used as an evacuation mattress.
